(19) United States
(12) Patent Application Publication
(10) Pub. No.: US 2003/0104512 A1
(43) Pub. Date: Jun. 5, 2003

(54) Biosensors for single cell and multi cell analysis

(76) Inventors: Alex R. Freeman, Plano, TX (US); Malgosia Wilk-Blaszczak, Coppell, TX (US)

Correspondence Address: Alex R. Freeman, 3733 Lowrey Way, Plano, TX, 75025, US

(21) Appl. No.: 10/013,017

(22) Filed: Nov. 30, 2001

(51) Int. Cl. C12Q 1/02

(52) U.S. Cl. 435/29

(57) ABSTRACT

The present invention relates to a structure comprising a biological membrane and substrate with fluidic network, an array of membranes and an array of fluidic networks in substrate, a high throughput screen, methods for production of the membrane, substrate structure, and a method for interconnected array of substrate structures and a method for attaching membranes to structure, a method to electrically record events from the membranes and a method to screen large compound library using the array. More particularly, it relates to biological cells and artificial cell membranes adhered to the substrate with a high electrical resistivity seal, a method to manufacture array configuration of such substrates, and a method to screen compounds using the membrane receptors such as ion-channels, ion pumps, & receptors.

### FIELD OF THE INVENTION

This invention generally relates to testing cells on microholes on membranes and more specifically to micro array based positioning of cells, recording the ion flux passing through the membrane of the cells using electrodes on the substrates.

### BACKGROUND OF THE INVENTION

### PRIOR ART

Ion channels are membrane proteins that act as gated pathways for the movement of ions across cell membranes and have crucial functions in cell physiology. A number of diseases are associated with defects in ion channel function (channelopathy). Ion channels are such an ubiquitous and essential component of the cell that defects in ion channel function have profound physiological effects. Recent listings of voltage-gated ion-channel compounds nearing or in clinical development reflect an ever growing level of investment in voltage-gated ion-channel R&D.

Voltage-gated ion channels are emerging as a major target class of increasing importance to pharmaceutical companies. They address a wide variety of diseases pertaining to the central nervous system (CNS), cardiovascular, nervous disorders, and metabolism. Voltage-gated ion channels play a critical role in shaping the electrical activity of neuronal and muscle cells, and in controlling the secretion of neurotransmitters and hormones through the gating of the calcium ion entry. Large families of voltage-gated Na⁺, K⁺ and Ca²⁺ ion channels have been defined using electrophysiological, pharmacological and molecular techniques. In the quest to identify novel compounds, drug companies are increasingly relying on High Throughput Screening (HTS) approaches.

### Introduction to Electrophysiology and Patch-Clamp Technique

Electrophysiology technique allows the recording of electrical events in electrically excitable tissues and cells. All of the electrical events in the living systems stem from the function of ion channels. Currently a manual electrophysiology technique called patch clamp allows for real time monitoring of ion channel function while simultaneously manipulating the excitability of the cell and allowing for direct physical access both to the interior and exterior of the cell membrane. In addition to the acquisition of very high levels of very high resolution information (possible monitoring of single molecule function with the time resolution below 1 millisecond) the delivery of drugs is possible from both sides of the cell membrane. The goal of the experiment is to measure the ion flux through the cell membrane between the cell interior and the bathing solution. This is achieved by observing ion-flux that is detected by an electrode inside a pipette, in fluidic connection with interior of the cell. The cell membrane makes a tight electrical seal of gigaohm impedance around the pipette. A reference electrode is immersed in the bathing solution outside of the cell. The DC current flowing between these two electrodes is the total ion flux crossing the membrane.

An ionic current flowing across the cell membrane inherently alters the membrane potential. That is why it is necessary to use a voltage clamp to hold the membrane potential constant when recording macroscopic or single-channel currents. The current flowing through the membrane at any particular potential can then be measured. This current is the sum of the ionic current, which represents current flow through open ion channels, and the capacitive current, which is largely due to the charging of the membrane capacitance. The capacitive current flows only while the membrane voltage is changing. When the cell membrane potential is held constant there will be no capacitive current, and the ionic current will be the same as the membrane current flowing through ion channels. This is one advantage of the voltage clamp method. A further advantage is that it prevents the resultant changes in membrane current from influencing membrane potential and activation of voltage gated channels (regenerative potential response such as the action potential). This technique permits measurement of the effect of changes in membrane potential on the conductance to individual ion species. By imposing the appropriate voltage on the cell membrane, ionic conditions and channel blockers, the exact channel species can be determined and then investigated.

Despite the above prior art, a high throughput platform does not exist to implement the above protocols. The following is the current status of efforts to implement high throughput screening. The common drawback in all these techniques is that the complete method as described above cannot be implemented in their systems.

### Limitations of Existing Methods

WO96/13721 describes a semi-automated apparatus for patch clamp technique. It discloses patch clamp apparatus utilizing an autosampler coupled to a conventional patch clamp. The throughput of this system is far below the high throughput needed for drug discovery and development. This technique is also not parallel and does not offer simultaneous testing of cells.

WO99/66329 describes a perforated substrate for sealing cell membrane for testing. The method of generating the perforation is by shining a laser on the substrate, which burns off the selected area of the substrate. The cells are made to culture and close the perforations. The limitations of this technique are varied. Laser ablation cannot generate under a 10 micron diameter hole reliably and the size variations of the hole opening in the substrate would present significant variations in patch clamp tests. Conventional patch clamp technique uses less then a 2 micron diameter hole, which presents even more significant problems for laser perforation. Secondly, the technique is not a single cell technique, instead cells are allowed to be cultured and the cell monolayer makes a tight junction between the neighboring cells. The sealing is achieved not between the hole and the cell membrane, but rather between the hole and the sheet layer of cultured cells. This is not a patch clamp technique in the traditional sense since single cell recording cannot be achieved. This technique also does not provide a way to position the cells on top of the holes, since conventional dispensing techniques cannot be used for precision positioning below 50 microns. Also, the discussion of perforating the cell membrane has not been discussed and is considered to pose significant challenge in implementing the high throughput screening as claimed.

In addition, the differences in the structural details are numerous. The membrane in the current invention overhangs on top of a microfluidic channel. The perforation is only on the membrane and the substrate has a different pattern of perforation of considerably larger size than the perforation in the membrane. The membrane is attached to the substrate and independent patterns can be etched into the membrane and the substrate. The membrane is structurally weaker and substantially smaller in thickness than the substrate on which it is attached. The current invention also incorporates methods to position cells and perforate cells.

WO 00/34776 describes an interface patch clamping. In this method, the cells are suspended at the fluid-air interface due to the capillary forces of small openings. Pipette tips are moved up to penetrate these cells. Clearly, this technique is a direct scale up of the single patch clamp test with no room for parallel testing and batch manufacturing advantages of semiconductor processing.

Attempts have been made to incorporate ion channels in bilayers on Si/SiO₂ interfaces having 50-100 micron openings. The phospholipid bilayers are painted on the surface of the openings in silicon dioxide. MaxiK channels are reconstituted within the bilayer. Conventional patch clamp experiments are performed on these bilayers with fluid access on both sides of the bilayers. This approach to screening drugs introduces additional variations on the functional characteristics of the bilayers and channels. It also introduces the use of intact whole cells for drug screening, which is a much more viable method. No information on the biological pathways leading to the ion-channel triggering can be found out using the above method.

Another patent WO 99/31503 describes patch-clamp-based testing of cells on microstructured carriers. It uses electroosmotic pumping across a microhole through which the fluid is pushed, to position the cells. In the case of whole cells, electroosmotic fluid motion through the microhole does not guarantee positioning of the cell due to the adhesion of cells to the silicon dioxide surface. Both prevention of adhesion of the cell outside the area of the hole, and promotion of adhesion of the cell on the microhole is necessary to achieve successful cell positioning. Electroosmotic pumping alone is insufficient to maintain proper fluid driving forces since the pH of the medium needs to remain near 7.4, requiring excessive driving voltages which may have deleterious effects on the cell biology. Since pinhole free coating of oxide and nitride on silicon is difficult, electrical breakdown is a serious limitation at high voltages, which can be avoided by combined vacuum and electroosmotic flows. Local pH variations caused by the dissolved oxygen and carbon dioxide in the culture media can present variations in the fluid flows in the array configuration, disrupting the fluidic balance needed for array. No work was demonstrated for an array based patch clamp testing, where variations between the elements of array can occur, and a large array creates significant fluid management issues for filling each element of the array individually. In addition, silicon substrate can create noise problems that can be minimized by having dielectric layers. However, the required dielectric layers are not described. This patent has also failed to demonstrate that cells can be successfully tested in an array fashion. This clearly indicates that additional innovations are required for a successful patch clamp chip.

U.S. Pat. No. 5,981,268 describes the impedance measurement system for single cells on electrodes. This system does not provide a method to position the single cells, and in addition does not address the measurement for ion-channel flux using intra-cellular electrodes or provide any method of opening the cell membrane to measure ion exchange via the cell membrane. Similarly U.S. Pat. No. 4,054,646; U.S. Pat. No. 4,920,047; U.S. Pat. No. 5,187,096 by Giaever et al., describe impedance measurement of a layer of growing cells on large electrodes. The disadvantage of this method is that interference by other cells or uncovered areas of electrodes interfere with the measurements and no provision is made to position intra-cellular electrodes. Also, no cell permeability studies can be carried out either on single cells or layer of cells, and cell membranes were assumed to be of constant impedance with no ion flux crossing the membranes.

### SUMMARY OF THE INVENTION

Due to the above described limitations, there is a need for a combined intra-cellular and extra-cellular test system which can measure the ion fluxes crossing the cell membranes by intracellular electrodes, and the extra-cellular impedance measurements of single and multiple cells with a precise positioning of the cell on the electrodes.

The invention relates to measurement of the ion channel activity by positioning cells on microholes, which are in fluidic communication with microfluidic channels, both created in planar substrates. The invention relates to positioning cells on the microholes using combined hydraulic and electrokinetic forces. These forces are dynamically tuned across each microhole depending on the cell-microhole impedance seal. Further, the portion of the cellular membrane directly on the microhole is separated by hydraulic pressure and electric field. Electrodes placed on either side of the microhole detect the ion flux crossing the cell membrane. Various pharmaceutical compounds are interacted with the cells to study the effect on the ion channel kinetics.

### DESCRIPTION OF THE DRAWINGS

FIG. 1. is a cross-sectional view of the cell analysis system of the present invention with the necessary fluidics and electrodes according to an embodiment of the present invention;

FIG. 2. is a cross-sectional enlarged view of a single cell adhesion on the opening of the substrate membrane;

FIG. 3. is a cross sectional view of the substrates for cell monolayer impedance detection;

FIG. 4. is an independent input array of test sites and fluidics scheme;

FIG. 5. is the common input array of test sites and fluidics scheme;

FIG. 6. is the exploded view of the various layers of the structure;

FIG. 7. A-G are manufacturing cross sectional views;

FIG. 8. is the cross sectional view of combined optical and electrical analysis of biological material;

FIG. 9. is the microscopic view of the structural membrane opening;

FIG. 10. is the structural membrane with cell culture media;

FIG. 11. is the view of cell positioned on the membrane opening;

FIG. 12. is the impedance without cells on the membrane opening;

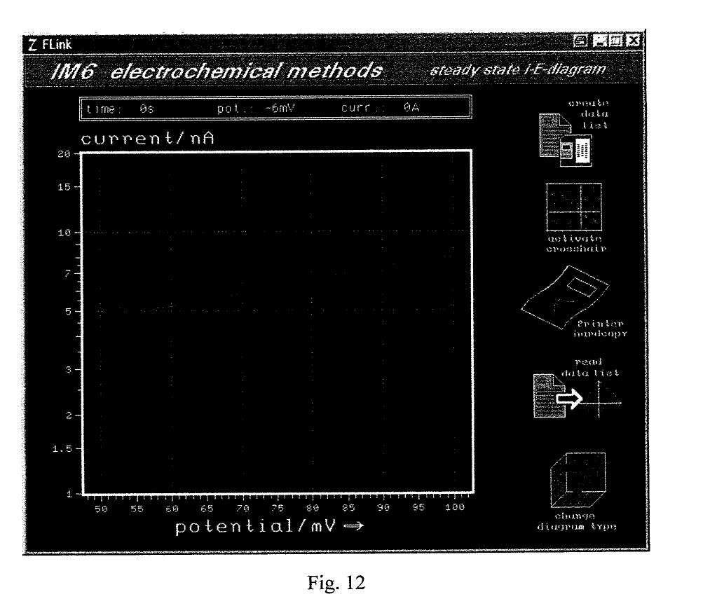

FIG. 13. is the impedance with cell positioned on the structural membrane opening.

### DESCRIPTION OF THE PREFERRED EMBODIMENTS

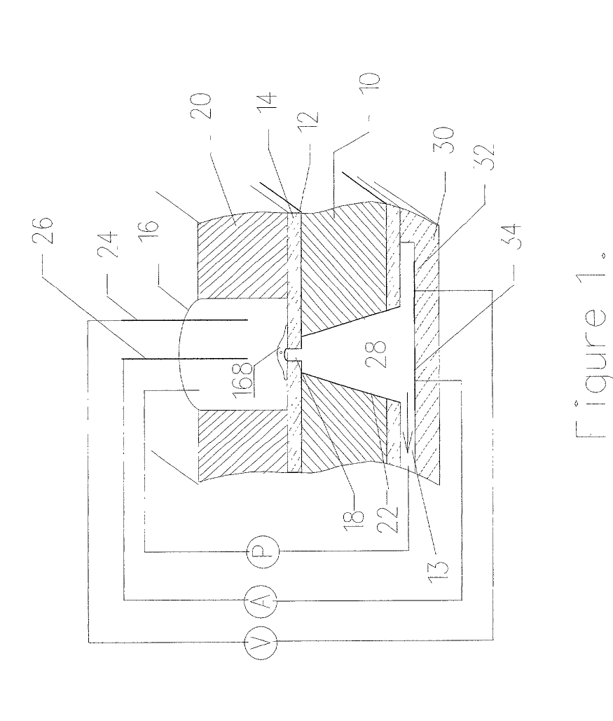

A preferred embodiment of the current invention as shown in FIG. 1 includes a substrate 10, which defines a through hole 28 in the substrate. The substrate has a membrane 14 disposed over it with a microhole 18 positioned directly over through hole 28. In another embodiment, membrane 14 has controlled porosity rather than the patterned microholes. Membrane 14 may be a thin film membrane. Membranes of thin films can be integrally manufactured by various thin film technologies known in the art such as chemical vapor deposition, spin coating, plasma deposition, etc. Thin film membrane materials include silicon nitride, silicon oxide, porous polysilicon, photoresists, polymers and gels. Membrane 14 can also be a thick film formed by thick film deposition technologies such as screen printing, immersion coating, doctor blade process, spray coating etc. The thick film materials include any of the porous gels, polymers, metal pastes, and organic inks. Preferably another thick film is the porous ceramics and especially glass frits. Glass frit exhibits controlled porosity ranging from 4 microns to 20 microns with a regular pattern. Such highly porous material offers a very large surface area for binding of the biological material. Due to the increased amount of bound biological material, a larger signal to noise ratio can be obtained. Silicon can also be made porous by electrochemical etching.

Wells are created in a second substrate 20 disposed adjacently to membrane 14. Substrate 20 has fluidic wells 16 ranging in diameter from 100 microns to 5 mm. The geometry of the wells is of any suitable shape to contain fluids. The geometry also allows electrodes 24 and 26 to be positioned either integral to second substrate 20 or physically placed at microwells 16 and moved from a series of microwells to another series of microwells. Substrate 20 can also include individual wells attached at each test site or integrally manufactured by any of the semiconductor processing techniques known to those skilled in the art. A third substrate 30 with a microfluidic channel network 13 is defined therein and coupled to substrate 10 in a leak tight fashion. Electrodes 24, 26, 32 and 34 are placed near membrane 14 either externally, or in a preferred embodiment, integrally manufactured on the monolithic substrates. One set of electrodes 26 and 34 across the membrane are used to drive the fluids across the membrane openings. The fluid motion occurs due to the electrical potential across the openings in the membrane through the thickness of membrane 14. Both electroosmosis and electrophoresis contribute to the total fluid motion across the membrane.

Another set of electrodes 24 and 32 are used to measure capacitance, impedance, conductance and current across the openings of the membrane. The electrodes maybe any one of or a combination of platinum electrodes, silver/silver chloride electrodes, gold electrodes, carbon thick film electrodes or externally positioned ion-selective electrodes.

Biological Substances Description:

Cells such as cardiac miocites, and neuronal cells contain transmembrane proteins, which form opening into the membrane allowing ions to pass from one side to the other. These proteins show ion specificity and some are open all the time called “leak channels”, while others have gates, which open based on the specific perturbance to cell membrane. The perturbation known to cause opening of the ion channels include cell membrane polarization change, mechanical stimulation, or the binding of signaling molecules.

Some of the cell types that can be dispensed in to the wells 16 are Chinese hamster ovary cells, primary neuronal tissue such as hippocampus, neuronal cells, dorsal root ganglia, muscle cells, miocites, cardiac tissue, epithelia, endothelia, liver cells, immune cells, and other suitable tissues. Cells genetically engineered to express various ion-channels or transporters may also be used. This includes transfecting the cells with cDNA or cRNA encoding of ion channel of interest and cloning the cells expressing the ion channel of interest. The ion channels that show specificity for sodium, potassium, calcium, chloride, hydrogen, and magnesium are used in the current embodiment. An artificial bilayer membrane may be attached to the substrate membrane and various ion channels maybe attached to the membrane. Stem cells, which upon further differentiation become various types of cells, are also possible biological materials that may be tested or analyzed using the current embodiment of the invention.

The current embodiment preferably interprets the junction resistance and ion transport mechanisms in epithelia. Epithelia are sheet of polarized cells joined together at their apical surfaces by tight junctions. Both absorption and secretion is occurs due to the net transport of electrolytes and non-electrolytes from lumen to plasma or from plasma to lumen. By measuring the electrolyte transport, and the changes in the transport mechanism induced by drugs, medium throughput drug screening and pharmacokinetic studies can be conducted.

Fluid containing cells of the above type in suspension is dispensed in to the wells 16 as shown in FIG. 1 for ion channel analysis. In another method, secretionary cells such as pancreas, bladder cells, pituitary cells, intestine cells are plated on the substrate membrane 14, 44 or 82 for the study of the biological pathways at work for secretion and regulation of secretion. In another method, retinal cells deposited on the structural membrane 14, 44 or 82 elicits an extra-cellular field potential, termed the electroretinogram. These signals are used to study the pharmacological impact on retinal cells.

Cell Positioning:

In the embodiment in FIG. 1, biological substances or cells are dispensed into wells 16 along with culture media. Combined electrokinetic and suction vacuum may be used to draw the fluid through microhole 18 and through hole 28 and connected channel network 13. This combination of pumping forces is essential since the pH of the medium cannot be adjusted suitably for the electroosmotic fluid flows desired. The impedance of the solution across membrane 14 is monitored continuously via the electrodes 24 and 32 as the fluid is drawn into channel network 13. When the cell covers microhole 18, the impedance goes up. An optimized protocol for applied drive voltage corresponding to the impedance of the cell sealing against the microhole is followed for achieving cell positioning and cell membrane sealing against microhole 18. Cell membrane 168 is perforated by a spike in the applied voltage across the substrate membrane opening. This spike in voltage momentarily increases the fluid flow, separating the membrane patch from the cell membrane.

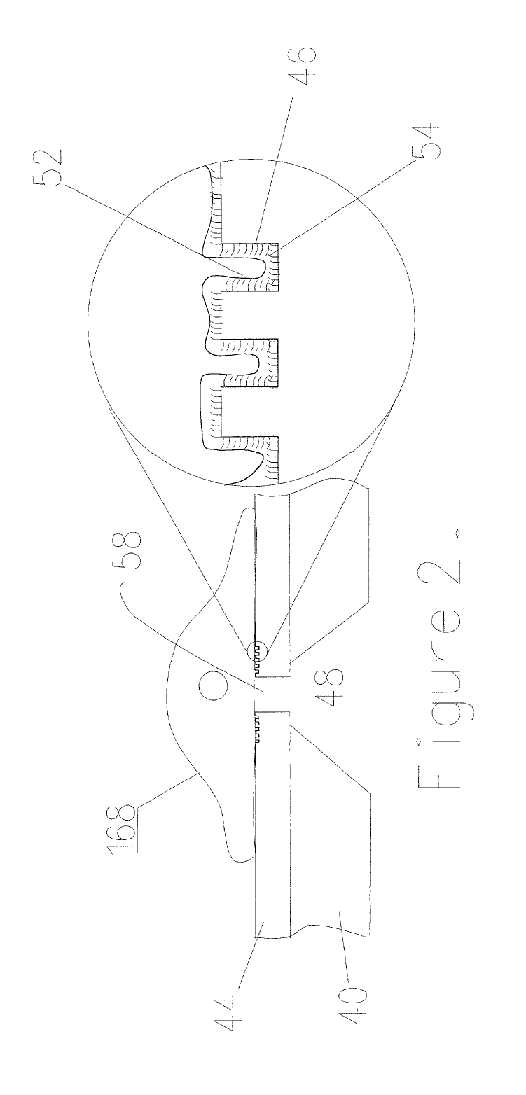

In another embodiment shown in FIG. 2, adhesion is improved by both chemical adhesion and geometrical optimization. A substrate 40 has a through hole 48 and a membrane 44 attached to the substrate. A membrane 44 has an opening 58 in fluidic concentration with through hole 48. Concentric to opening 58, micro grooves 46 of 0.1 micron to 2 microns width and 0.1 micron to 1 micron depth are etched. Cellular adhesion molecules such as polylylysine 54 are spotted around the opening. Preferably, cellular adhesion molecules such as adherin and cadherin, or synthetic molecules such as poly-lysine, fibronectin, collagen or gelatin mono or multilayers could be deposited either using photolithography techniques or micro contact printing around microhole 58. Additionally, hydrophobic molecules could be deposited in the remaining area around microgrooves 46 to prevent the cell from adhering to outside the areas of the microhole. When cell membrane 52 rests on microgrooves 46, because of the increased surface area due to the grooves, the membrane has a greater chance to be sealed with the polylysine or other adhesion molecules against the surface of the substrate membrane. This generates a tighter seal between the cell membrane and the substrate membrane. This tighter seal is important for the noise-free detection of the ion channel temporal dynamics on the single cells positioned on the opening 58. The current embodiment enables the membrane of the cell to fold inside of the grooves, further increasing the total sealing surface available.

Various pharmaceutical compounds or ion-channel agonists or antagonists maybe dispensed directly into microwell 16 in FIG. 1 or alternatively added to the liquid media of channel network 13. Either current clamping where the total current is maintained to be zero or voltage clamping, where the potential across two electrodes is maintained constant during the recording. In the current clamping, the membrane potential is recoreded and in voltage clamping, the ionic flux crossing the cell membrane is recorded.

In the preferred embodiment where the membrane (instead of having a single opening as described above) has a network of porosity, smaller molecules and fluid are to cross the membrane, but the larger molecules are blocked. The size of the molecules permitted to pass depends on the pore size and pore network. Membranes having 100 nanometer pore size to 10 microns in the case of glass frit are currently commercially available. Microcontact printing and photolithography are used to pattern the surface of the membrane to leave bare membrane surface areas for the fluid to communicate across the substrate membrane. This allows selective ion detection and increases the applicability of the current invention using porous membranes.

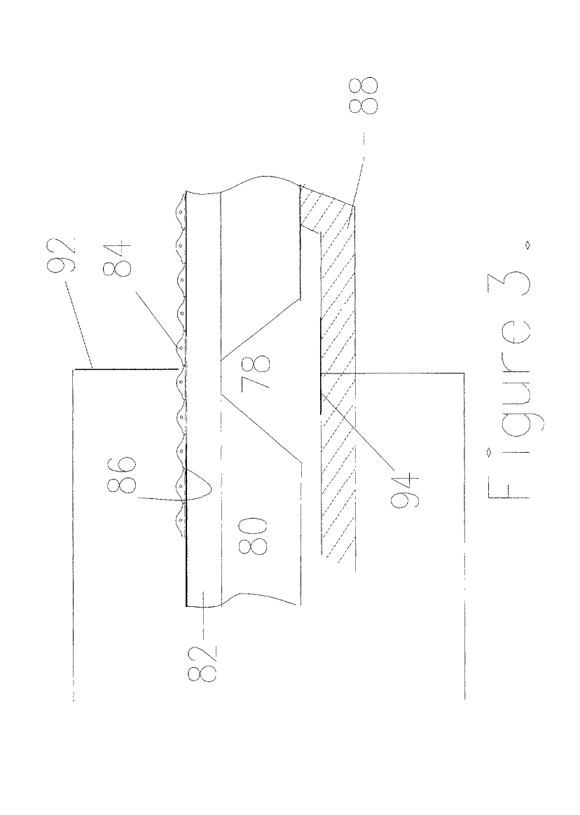

In yet another embodiment, as shown in FIG. 3, a membrane 82 is porous. This membrane can be patterned to contain hydrophobic polymer coated every where except near the region of through hole 78 as shown by 86. Cells 84 are plated on the porous membrane 82 on the substrate 80 and depending on the type of cells, tight monolayer or dispersed cells reside on the substrate membrane. Electrodes 92 and 94 are positioned on either side of membrane 82 as shown in FIG. 3. Cellular impedance and cell to cell junction impedance is measured across the substrate membrane. It is a further innovation of the current invention that capacitance across the same substrate membrane is measured and is modulated by the presence of the cell types, and number of cells on the porous part of the membrane.

Array Issues

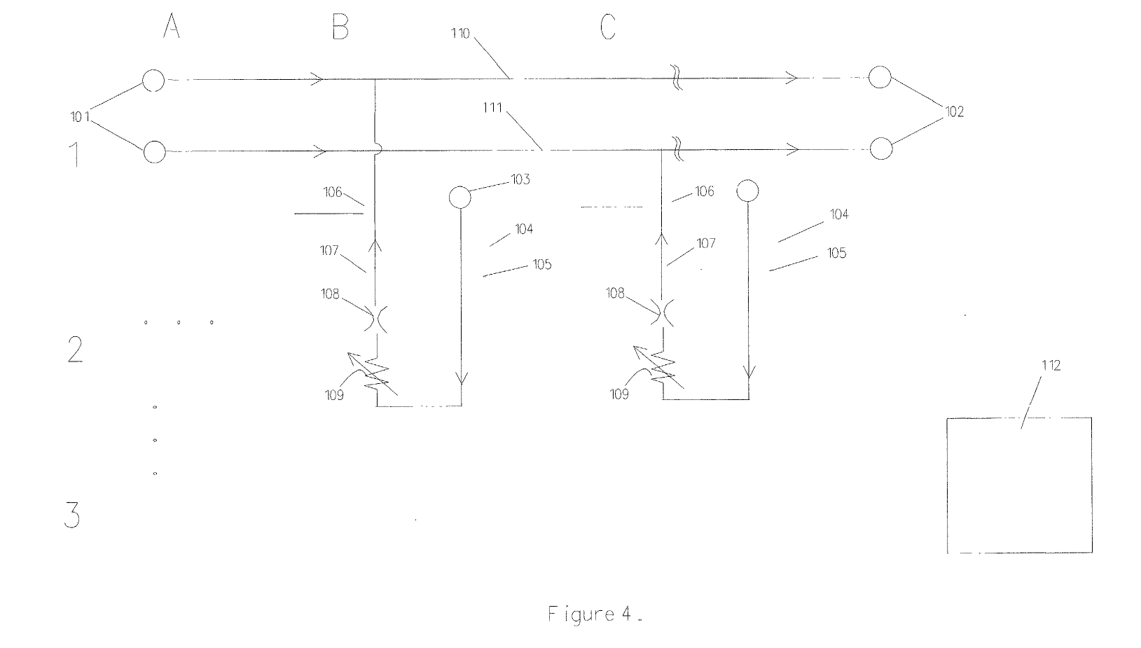

The above description is generally related to a single test site. In a preferred embodiment as shown in FIG. 4, an array of a plurality, e.g. 96 or 384, of test sites can be built on a single monolithic substrate. This allows simultaneous screening of a plurality of drugs or drug concentrations per monolithic substrate. Individual fluid microwells 103 are connected to each test site. Each test site includes multiple electrodes, preferably four electrodes 104, 105, 106 and 107, for each test site, two on each side of the substrate membrane. Fluid flow between wells 101 and 102 may be achieved by a combination of vacuum and electrokinetic flow. Since the media pH is close to 7.0 and cannot be changed, this pH requires excessive voltages to electrokinetically drive the fluids. A simulation conducted using CFDRC ACE software showed that a voltage of 5 KV is equivalent to a pressure of 1200 Pa for a 4 micron opening in the substrate membrane. This shows that the suction pressure required is very small compared to the electric fields needed to pump the fluid. Fluid in channels 110 and 111 connecting wells 101 and 102 are preferably achieved by vacuum suction with superposed electrokinetic pumping across each opening in the substrate membrane using the electrodes at the test site. In a preferred arrangement, only electrokinetic pumping is done between wells 101 and 102 with individual flow variations superposed on it across each test site. The embodiment in FIG. 4 allows independent test sites to be used in high throughput testing of a large library of pharmaceutical compounds.

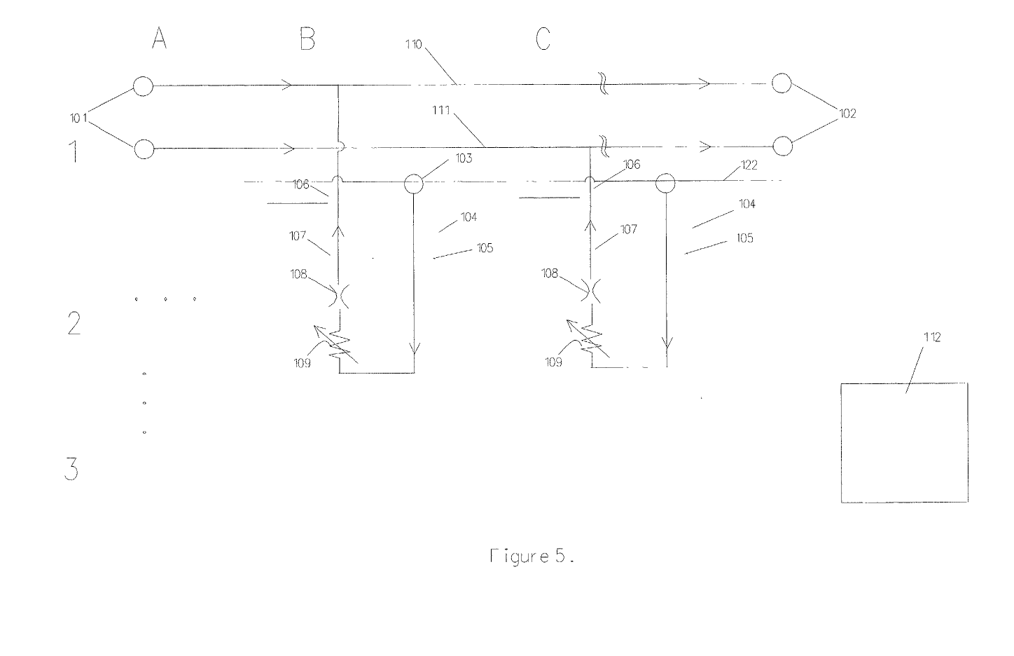

In another preferred embodiment as shown in FIG. 1, multiple membrane openings such as opening 18 and the corresponding through holes 28 in the substrate can be covered by a single microwell 16 in the substrate 20. The channel network 13 connecting through holes 28 is independent of each other. This allows multiple single cells subjected to the same media and drug concentrations to be tested simultaneously. The array arrangement for this is shown in embodiment in FIG. 5. The advantages of this approach over the arrangement in FIG. 4 is that, a single pressure pump can push the fluid through a line 122 in fluidic connection with microwells 103 of each test site, and individual test site flow variations can be controlled by the drive voltages across each opening in the substrate membrane. This approach of global hydraulic pressure with superposed electrokinetic pumping is one inventive concept of the current invention. This approach simplifies the number of vacuum or pressure pumps needed to an absolute minimum.

Electrodes 104, 105, 106, and 107 sense the currents and control the voltages individually in each of the test site to adjust the flow. The electrokinetic voltages are steadily increased until the point where the impedance registered across the opening the sutrate membrane reaches a predetermined high. Once a high value of impedance is detected, the voltage is quickly ramped down at that particular test site to a value where the cell membrane can be perforated due to the flow induced by the electrokinetics. After cell membrane perforation, and once the membrane has adhered to the substrate surface, the impedance goes up again and the voltage is further decreased. The voltage across the perforated cell is now clamped or fixed by transferring the electrons necessary to control the electrode reactions, which in turn maintain the voltage across the membrane constant. The above process sequence is repeated across all the test sites of the array. Then the ion flux is scanned serially across the chip, one test site at a time. These values are recorded for further analysis. Once the experiment is completed, the substrate is disposed and a new substrate is loaded from the storage rack.

Signal Processing and the Electronics

The parallel approach of the present invention increases the total throughput of the system. Once a cell membrane is opened, the voltage must be clamped and maintained at a fixed value. Therefore, a high speed, low noise multiplexer is necessary for voltage clamp, which is readily available. Such multiplexers are readily available. Cross talk between the patch clamp array can be minimized by data logging from each test serially rather than logging data simultaneously from all the channels. The total time for serial testing is longer than parallel testing, but may not be significant compared to cell positioning.

Low noise multiplexers need to be used in order to switch between the parallel patch clamp tests. For example, Agilent's E5250A multiplexer can accommodate 384 channels and switch between experiments without adding significant noise to the measurements. The speed of switching or the noise of switching are trade-offs. To keep the noise level low, a slow switching, low noise multiplexer would work well. The switching speed of E5250A is approximately 3.2 seconds. For a 96 sequential recording, this is a total time delay of about 5 minutes. This is not a significant throughput issue since the time for cell adhesion and positioning typically take much longer.

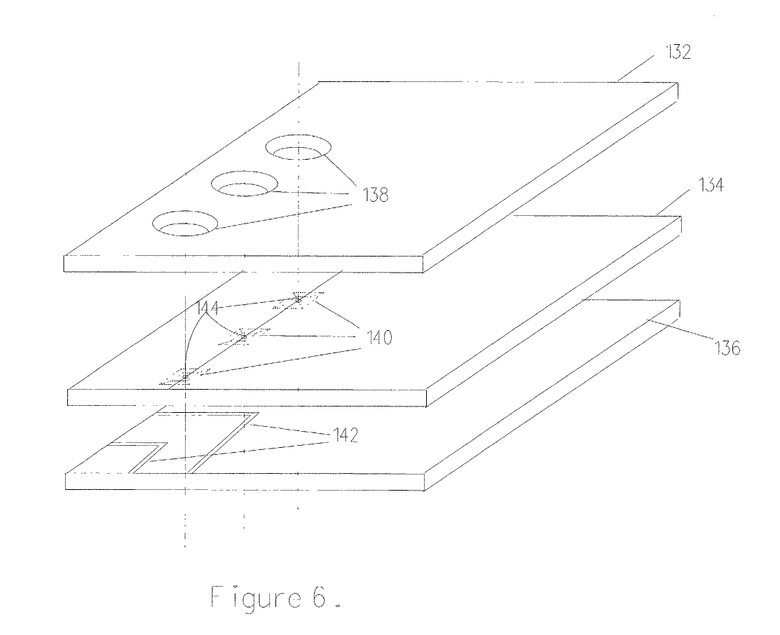

The embodiment of the present invention shown in FIG. 6 shows one of the arrangements for manufacturing the system. Three substrate layers 132, 134 and 136 are used for the formation of the entire network of test sites. Substrate 132 contains a plurality of microwells 138. Substrate 134 contains a plurality of membrane openings 144 and respective through holes 140 in the substrate. Substrate 136 contains channel networks 142 to connect the test sites. The electrodes are either integrally manufactured on any of the above substrates or placed on the test sites or a combination of both. The substrates can be of any material such as polydimethly siloxane, silicones, polymers, photoresists, silicon, glass, quartz, etc.

Manufacturing

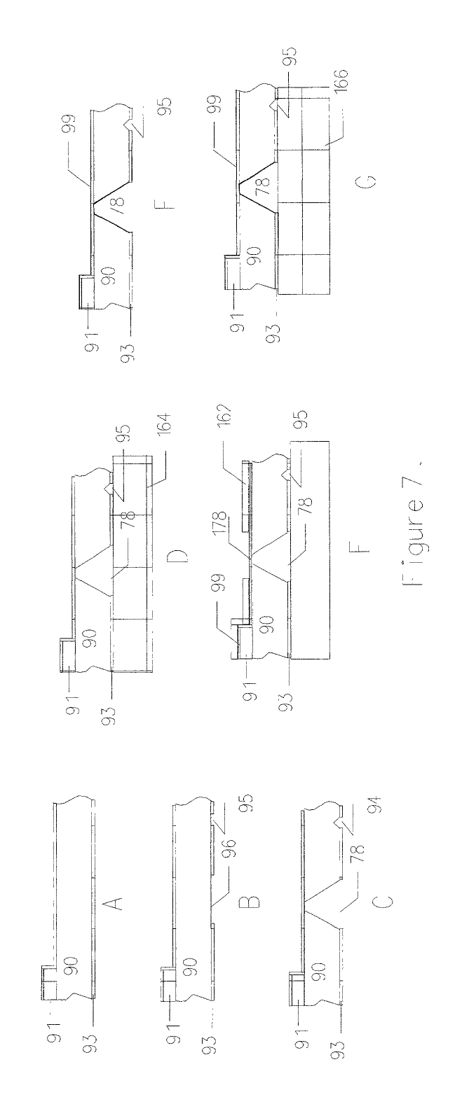

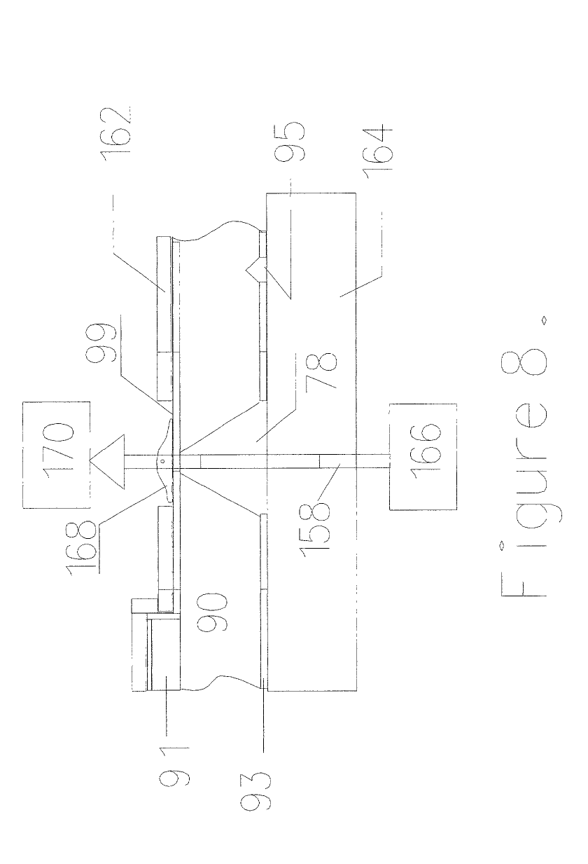

Referring to the process sequence in FIG. 7.A-G, the manufacturing sequence is described further. Semiconductor grade 100 double sides polished preferably 525 micron thick silicon wafers maybe used as starting materials for substrate 90. Thin, approximately 1000A, thermal oxide is grown on the silicon wafer. Low stress nitride, 93 of approximately 7200A is grown on top of the thermal oxide. Various electronics semiconductor processing is done on the substrate 90 to create the circuits 91 without the final metallization. This processed wafer is further used as the starting substrate. The front side of the wafer is etched using plasma etching for thinning of the various oxide layers to give a final nitride and oxide layers on the silicon substrate as in FIG. 7.A. Both the front and backside of the wafer are patterned (FIG. 7.B) and the nitride oxide layer is etched as in 95 and 96 using plasma etching. The exposed silicon substrate is then wet etched using TMAH (FIG. 7.C) to obtain the microfluidic passages 78 and 94. Further thin thermal oxide is grown on the silicon substrate to insulate the silicon substrate from large voltages that would be applied during the testing. The silicon wafer is in a preferred embodiment, bonded to a substrate of pyrex, glass, quartz or silicon substrates 164 as in FIG. 7.D. Metal coating 99 to connect the electronics 91 to the biosensors is carried out by patterning of various metals such as gold, platinum, aluminum and chromium. This can be done on preferably both sides of the wafer as also shown in FIG. 7.F. From here, low temperature bonding techniques can be used to bond substrate 166 to the substrate 90 to form the closed channels 78 and 95 as in FIG. 7.G. Substrates 164 and 166 can in addition have etched fluidic network to connect the microfluidic channels 78 and 95 leading to very versatile fluidic network. Structures 8.E or G lead to a device where thin membrane 178 is suspended above the microfluidic channel 78. The thin membrane is transparent and with less than 1500 angstroms of gold, the membrane with the gold layer is sufficiently transparent to do optical detection of biological material placed on top of the membrane 178. The final passivation layer 162 protects the electronics and the metallization. The current manufacturing technique gives a unique process to integrate microfluidics 78, 95, electronics 91, optical incidence 166 and optical detection 170 on a single chip platform as shown in FIG. 8 by the optical path 158. The thin membrane structure is sufficiently strong and is transparent with integrated electrodes, allowing the optochemical detection of biological material 168 with on-chip electronic processing. This platform is not restricted to any one type of biological material and allows a wide detection for fluorescent, chemiluminiscent, electrochemical, colorometric detection, analysis and signal processing.

### EXAMPLE

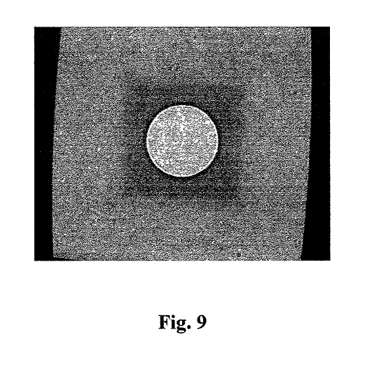

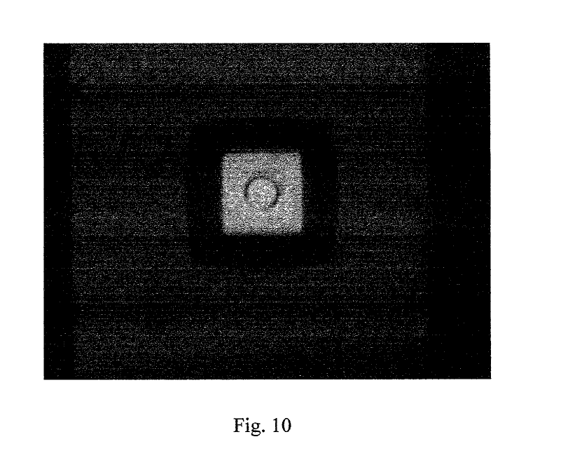

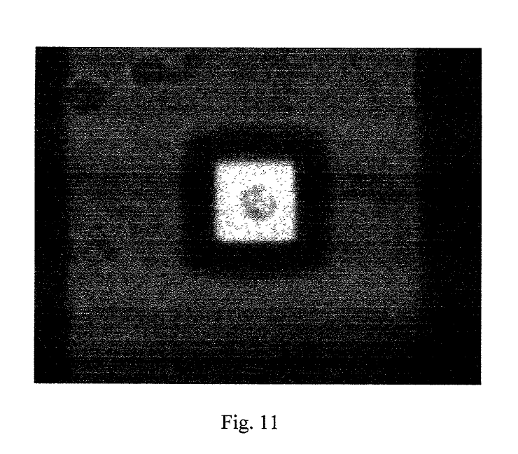

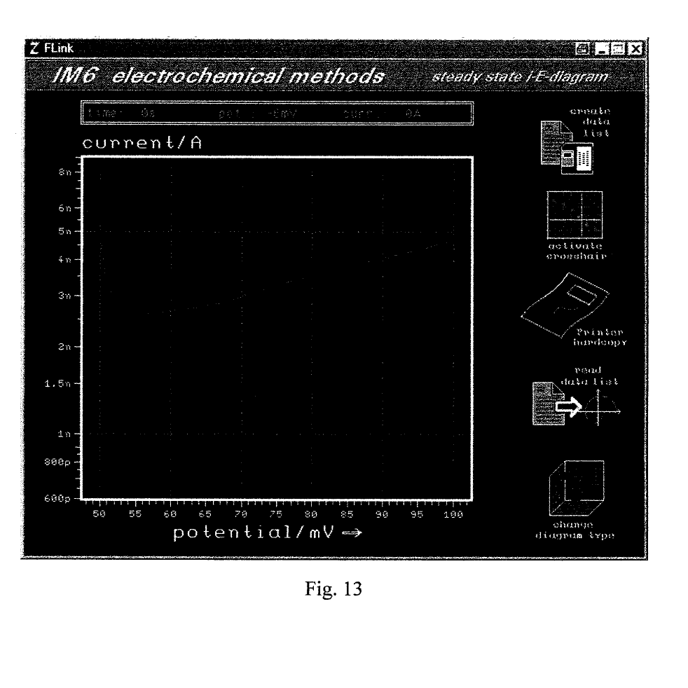

FIG. 9 shows an embodiment of the resulting device as manufactured by the above methods. A silicone substrate 20 is glued to the substrate 10 to create the well 16 on top of the membrane opening 18. Similar substrate 30 is used to create the channel network underneath the through hole opening 28. NG108-15 cells with the culture medium are dispensed into the wells 16. Both fluidic only pumping and electrokinetic pumping have been demonstrated with these cells. The cells are positioned on the substrate membrane opening in both the above pumping cases. FIG. 10 and 11 show the chip with and without the cells positioned on the substrate membrane opening. External silver/silver chloride electrodes are used to detect the impedance of the liquid with and without the cell. 213 shows the impedance without the cell and FIG. 13 shows the impedance with the cell positioned on the substrate membrane opening. The results clearly show the presence of the cell on the microhole 18 due to the change in impedance. This change in impedance grows as the cell seals the microhole 18 completely.

What is claimed is:

1. A structure comprising:
a substrate having a microfluidic channel;
a substrate membrane disposed over the substrate and in fluidic connection with the microfluidic channel, and further having at least one opening operable to allow passage of molecules having a set of predetermined characteristics;
first and second electrodes disposed on both sides of the substrate membrane operable to detect molecular transport across the substrate membrane and a biological substance deposited thereon.

2. The structure, as set forth in claim 1, wherein the at least one opening of the substrate membrane comprises a plurality of pores in the substrate membrane.

3. The structure, as set forth in claim 1, further comprising an electric field applied across the substrate membrane operable to displace a liquid retained on one side of the substrate membrane to the other side of the substrate membrane via the at least one opening.

4. The structure, as set forth in claim 1, where the biological substance is selected from the group consisting of individual cells, cells grown in a monolayer, tissue samples, artificial lipid bilayers with embedded proteins, cells from animals, plants and humans, miocites, ion-channel expressed oocytes, CHO cells, muscle cells, epithelia and immune cells.

5. The structure, as set forth in claim 1, where the biological substance further comprising a biological membrane having ion-channel proteins or transporter molecules and adhering to the at least one opening of the substrate membrane, making a tight electrical and fluidic seal.

6. The structure, as set forth in claim 1, wherein the substrate membrane comprises at least one opening operable to allow passage of molecules depending on their size, ionic charge and shape.

7. The structure, as set forth in claim 1, where the at least one opening of the substrate membrane comprises a microhole having a diameter ranging between 1 to 10 microns. inclusively

8. The structure, as set forth in claim 1, to measure impedance of cells positioned on the at least one opening.

9. The structure, as set forth in claim 7, to measure impedance of cells positioned on the microhole.

10. The structure, as set forth in claim 1, further comprising an array of openings formed in the substrate membrane disposed over an array of through holes formed in the substrate.

11. The structure, as set forth in claim 1, wherein the substrate membrane comprises a porous gel.

12. The structure, as set forth in claim 1, wherein the substrate membrane comprises glass frit.

13. The structure, as set forth in claim 1, wherein the substrate membrane comprises silicon nitride thin film.

14. The structure, as set forth in claim 5, wherein the substrate membrane further comprises a cell adhesion coating operable to promote adhesion of the biological membrane to the substrate membrane.

15. The structure, as set forth in claim 1, wherein the first and second electrodes are constructed of an ion-selective material or compound material selected from a group consisting of gold, platinum, and silver/silver chloride.

16. The structure, as set forth in claim 1, wherein the biological membrane is constructed of gland cells selected from a group consisting of pancreas, intestine, and gallbladder.

17. The structure, as set forth in claim 7, further comprising at least one micro-groove formed around the microhole.

18. The structure, as set forth in claim 7, further comprising 1 to 20 micro-grooves having 0.1 micron to 2 microns width and 0.1 micron to 1 micron depth formed around the microhole

19. The structure, as set forth in claim 7 or 18, wherein the substrate membrane further comprises a cell adhesion coating around the microhole and covering a diameter of 10 microns to 1000 microns.

20. The structure, as set forth in claim 10, further comprising an electromechanical force operable for steering an array of fluids into and out of the array of through holes in the substrate.

21. The structure, as set forth in claim 20, wherein the electromechanical force comprises at least one of an electrokinetic force and a vacuum force.

22. The structure, as set forth in claim 1, wherein at least one of the first and second electrodes is formed integrally on the substrate membrane.

23. A method, comprising:
dispensing biological substance on a substrate membrane having predefined porosity, the substrate membrane being disposed over a substrate having a microfluidic channel in fluidic communication with the substrate membrane;
applying an electrical potential across the substrate membrane; and
detecting and measuring a charged species flux across the substrate membrane.

24. The method, as set forth in claim 23, where the biological substance is selected from the group consisting of individual cells, cells grown in a monolayer, tissue samples, artificial lipid bilayers with embedded proteins, cells from animals, plants and humans, miocites, ion-channel expressed oocytes, CHO cells, muscle cells, epithelia and immune cells.

25. The method, as set forth in claim 23, further comprising positioning at least one cell in a microhole formed in the substrate using electrokinetic fluid pumping.

26. The method, as set forth in claim 23, further comprising positioning at least one cell in a microhole formed in the substrate by applying a force selected from at least one of a large potential across the microhole and a vacuum.

27. The method, as set forth in claim 23, further comprising perforating a cell membrane that is positioned on the microhole by an applied force.

28. The method, as set forth in claim 23, further comprising perforating a cell membrane that is positioned on the microhole by applying at least one of an electric field and a vacuum across the microhole.

29. The method, as set forth in claim 25, further comprising supplying the at least one cell with ion-channel agonists or antagonists on either side of the cell membrane.

30. The method, as set forth in claim 25, further comprising transfecting the at least one cell with cDNA or cRNA encoding of ion channels of interest and cloning of cells.

31. The method, as set forth in claim 25, further comprising immersing the at least one cell in physiological solutions.

32. The method, as set forth in claim 25, further comprising optical incidence and optical detection of cells from either side of the substrate. The optical methods are any of exposing the biological substance to an optical source;
and detecting an optical property of the biological substance.

33. The method, as set forth in claim 25, further comprising applying an electrical potential to temporarily open the pores on the cell membrane.

34. The method, as set forth in claim 23, wherein measuring comprises measuring cell membrane conductance.

35. The method, as set forth in claim 23, wherein measuring comprises measuring cell membrane impedance.

36. The method, as set forth in claim 23, wherein measuring comprises measuring cell membrane ion channel current.

37. The method, as set forth in claim 23, wherein measuring comprises measuring cell-to-cell junction resistance.

38. The method, as set forth in claim 23, wherein dispensing biological cells comprises dispensing biological cells on an array of substrate membranes, the array of substrate membranes being disposed over a substrate having respective microfluidic channels in fluidic communication with the respective substrate membranes, and performing an array of tests with pharmacological compounds for the purpose of high throughput testing.

39. The method, as set forth in claim 23, further comprising positioning at least one cell in a microhole formed in the substrate by applying a large potential across the microhole using a pair of electrodes, measuring biological membrane to microhole seal resistance, and to vary the applied potential to adjust the position of the cells in response to the measured seal resistance.

40. The method, as set forth in claim 23, further comprising:
culturing retinal cells on the substrate membrane;
activating the cells with light; and
recording ion concentrations of sodium, potassium, calcium and chlorine.

41. The method, as set forth in claim 38, further comprising using a multiplexer coupled to the substrate to facilitate array-based high throughput drug screening.

42. The method, as set forth in claim 39, further comprising adjusting fluid flow in a throughhole formed in the substrate and in fluid communication with the microhole by applying a large potential across the microhole using a pair of electrodes, measuring biological membrane-to-microhole seal resistance, and to vary the applied potential to adjust the position of the cells in response to the measured seal resistance.

43. The method, as set forth in claim 42, further comprising:
measuring impedance across the throughhole; and
adjusting the fluid flow in each through hole of the array by varying the drive potential in an array in response to the measured impedance across the corresponding through hole.

44. The method, as set forth in claim 27, further comprising:
measuring impedance across the throughhole;
controlling the electric fields or suction fluid forces to perforate the cell membrane in response to the measured impedance across the throughhole.

45. A method of forming, comprising:
chemically depositing a thin film of thermal oxide and nitride on both sides with a first side of a substrate having an electronic circuit;
patterning the thin film on the second side of the substrate and using this patterned thin film as a mask;
creating an opening on the second side of the substrate to form a suspended thin film membrane on the first side allowing transmission of light there through;
forming an electrode pattern on the first side, the electrode pattern being coupled to the electronic circuit.

46. The method as claimed in 45, forming prior to forming an electrode pattern further comprising;
forming a second substrate on the patterned second side of the first substrate, thereby creating a microfluidic channel in fluid communication with the suspended membrane.

47. The method as claimed in 45:
forming a second substrate on the patterned second side of the first substrate, thereby creating a microfluidic channel in fluid communication with the suspended membrane.

48. The method as claimed in 45:
forming an electrode pattern on the second side;
forming a second substrate on the patterned second side of the first substrate, thereby creating a microfluidic channel in fluid communication with the suspended membrane.

49. The method as claimed in claims 46, 47 and 48:
attaching the second substrate by gluing with adhesive agent.

50. The method as claimed in claims 46:
attaching the second substrate by heating and applying pressure on both first and second substrates.

51. A method according to claim 16, where the effect of secretion from the cells on the ion-channel kinetics is explored and the method of modifying the secretion with drugs is studied.
